(19) United States
(12) Patent Application Publication
(10) Pub. No.: US 2007/0255242 A1
(43) Pub. Date: Nov. 1, 2007

(54) Wetness-sensing absorbent articles

(75) Inventors: Thomas M. Ales III, Neenah, WI (US); Andrew M. Long, Appleton, WI (US)

Correspondence Address: KIMBERLY-CLARK WORLDWIDE, INC.;Catherine E. Wolf, 401 NORTH LAKE STREET, NEENAH, WI, 54956, US

(73) Assignee: Kimberly-Clark Worldwide, Inc.

(21) Appl. No.: 11/414,035

(22) Filed: Apr. 27, 2006

(51) Int. Cl. A61F 13/20

(52) U.S. Cl. 604/361

(57) ABSTRACT

A method for assisting a caregiver who is addressing a lack of urination control in a subject includes manufacturing a plurality of wetness sensing systems, each including a plurality of wetness sensing components; providing the wetness sensing systems to a plurality of geographically-distributed retail outlets; and increasing consumer awareness of the wetness sensing systems by promoting the wetness sensing systems through a plurality of channels. Also, a system for increasing consumer convenience in acquiring wetness sensing components includes a first manufacturing facility for producing wetness sensing absorbent articles; a second manufacturing facility for producing signaling devices compatible with the wetness sensing absorbent articles; and at least 25 retail outlets, each receiving at least one of the wetness sensing absorbent articles and at least one of the signaling devices, wherein a wetness sensing absorbent article purchased at one retail outlet is compatible with a signaling device sold at another retail outlet.

### BACKGROUND OF THE INVENTION

Absorbent articles such as diapers, training pants, incontinence products, feminine hygiene products, swim undergarments, and the like conventionally include a liquid permeable body-side liner, a liquid impermeable outer cover, and an absorbent core. The absorbent core is typically located in between the outer cover and the liner for taking in and retaining liquids (e.g., urine) exuded by the wearer.

The absorbent core can be made of, for instance, superabsorbent particles. Many absorbent particles, especially super absorbent particles, are so efficient at absorbing liquids that it is sometimes difficult to tell whether or not the absorbent article has been insulted with a body fluid.

Accordingly, various types of moisture or wetness indicators have been suggested for use in absorbent articles. The wetness indicators may include alarm devices that are designed to assist parents or attendants to identify a wet diaper condition quickly upon insult. The devices produce either a visual or an audible signal.

In some embodiments, for instance, inexpensive conductive threads or foils have been placed in the absorbent articles. The conductive materials serve as conductive leads for a signaling device and form an open circuit in the article that can be closed when a body fluid, such as urine, closes the circuit. In these embodiments, although the absorbent articles may be disposable, the signaling devices are not. Thus, the signaling devices are intended to be removed from the article and reattached to a subsequent article.

Problems, however, have been encountered in using such articles for training and/or notification purposes in that executions of the concept are very limited in their availability, convenience, and efficacy, which translate to a loss in benefit to the consumer. In addition, systems are limited in their flexibility in that they cannot be adapted for different training situations or stages.

### SUMMARY OF THE INVENTION

As caregivers undertake training and/or notification programs to address toilet training, enuresis control, or incontinence monitoring, which apply across demographics, those caregivers would benefit by gaining greater access to products that are specifically designed to enhance the effectiveness of such training and/or notification programs.

Existing personal wetness sensors are either not reduced to practice or are facilitated by an entity that lacks the means for delivering a true solution to the customer. Because wetness sensing aids used for potty training, for example, have been demonstrated to be efficacious as training aids, it is clear that caregivers would benefit by having easy access to efficacious and reasonable-priced wetness sensing systems.

The invention described herein solves the problems described above and provides an increase in efficacy in using wetness sensing absorbent articles by increasing the availability, awareness, consistency, and caregiver confidence of wetness sensing systems. In general, the present disclosure is directed to wetness sensing absorbent articles with easy-to-use signaling devices. The signaling device, for instance, may be configured to indicate to a user that a body fluid is present in the wetness sensing absorbent article.

For example, in one embodiment, the invention includes a method for assisting a caregiver who is addressing a lack of urination control in a subject, the method including manufacturing a plurality of wetness sensing systems, each including a plurality of wetness sensing components; providing the wetness sensing systems to a plurality of geographically-distributed retail outlets; and increasing consumer awareness of the wetness sensing systems by promoting the wetness sensing systems through a plurality of channels.

In another embodiment, the invention includes a system for increasing consumer convenience in acquiring wetness sensing components, the system including a first manufacturing facility for producing wetness sensing absorbent articles; a second manufacturing facility for producing signaling devices compatible with the wetness sensing absorbent articles; and at least 25 retail outlets, each receiving at least one of the wetness sensing absorbent articles and at least one of the signaling devices, wherein a wetness sensing absorbent article purchased at one retail outlet is compatible with a signaling device sold at another retail outlet.

### BRIEF DESCRIPTION OF THE DRAWINGS

The foregoing and other features and aspects of the present invention and the manner of attaining them will become more apparent, and the invention itself will be better understood by reference to the following description, appended claims and accompanying drawings, where:

FIG. 1 is a rear perspective view of one embodiment of an absorbent article;

FIG. 2 is a front perspective view of the absorbent article illustrated in FIG. 1;

FIG. 3 is a plan view of the absorbent article illustrated in FIG. 1 with the article in an unfastened, unfolded and laid flat condition showing the surface of the article that faces away from the wearer;

FIG. 4 is a plan view similar to FIG. 3 illustrating the surface of the absorbent article that faces the wearer when worn and with portions cut away to show underlying features; and

FIG. 5 is a perspective view of the embodiment shown in FIG. 1 further including one embodiment of a wetness sensing system.

Repeat use of reference characters in the present specification and drawings is intended to represent the same or analogous features or elements of the present invention.

### DETAILED DESCRIPTION OF THE INVENTION

It is to be understood by one of ordinary skill in the art that the present discussion is a description of exemplary embodiments only, and is not intended as limiting the broader aspects of the present invention.

The present disclosure is generally directed to wetness sensing absorbent articles adapted to be attached to a signaling device that may be configured to indicate the presence of a body fluid in the absorbent article or other changes in the condition of the product or wearer. The absorbent article may be, for instance, a diaper, a training pant, a pre-fastened pant, a swimming pant, an incontinence product, a feminine hygiene product, a medical garment, a bandage, and the like. Wetness sensing absorbent articles may include an open circuit that becomes closed when a conductive fluid, such as a body fluid, is present in between a pair of conductive leads. Alternatively, wetness sensing absorbent articles may include a closed circuit that becomes open when a fluid, such as a body fluid, is present. Generally, the wetness sensing absorbent articles containing the circuit are disposable meaning that they are designed to be discarded after a limited use rather than being laundered or otherwise restored for reuse.

The circuit contained within the wetness sensing absorbent articles of the present disclosure is configured to be attached to a signaling device. The signaling device can provide power to the circuit while also including some type of audible, visible and/or electromagnetic signal that indicates to the user the presence of a body fluid. Although the wetness sensing absorbent article may itself be disposable, the signaling device may be reusable from article to article. In this regard, the present disclosure is particularly directed to different types of attachment mechanisms that allow easy connection between the circuit in the wetness sensing absorbent article and the signaling device.

As described above, the circuit in combination with the signaling device may be configured to indicate the presence of a body fluid contained within the wetness sensing absorbent article. The particular targeted body fluid may vary depending upon the particular type of wetness sensing absorbent article and the desired application. For instance, in one embodiment, the wetness sensing absorbent article comprises a diaper, a training pant, or the like and the signaling device is configured to indicate the presence of urine. Alternatively, the signaling device may be configured to indicate the presence of a metabolite that would indicate the presence of a diaper rash. For adult incontinence products and feminine hygiene products, on the other hand, the signaling device may be configured to indicate the presence of a yeast or of a particular constituent in urine or menses, such as a polysaccharide.

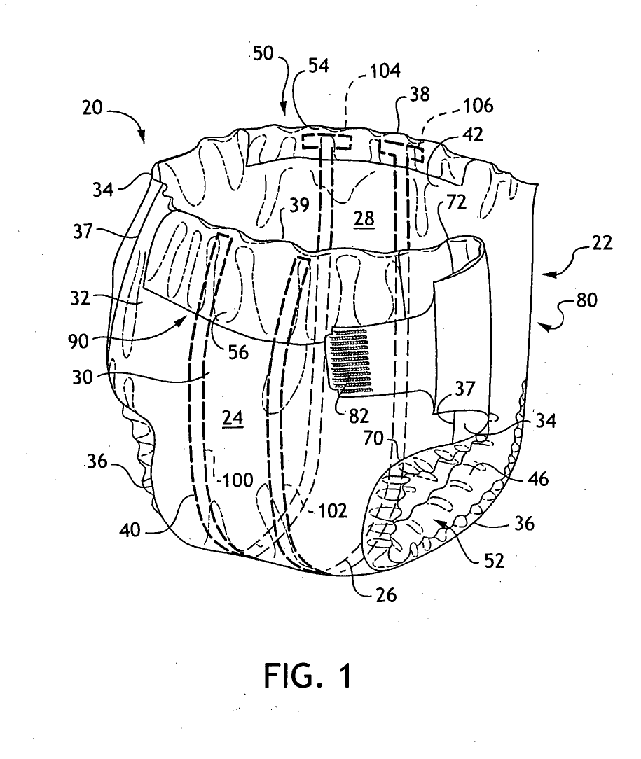

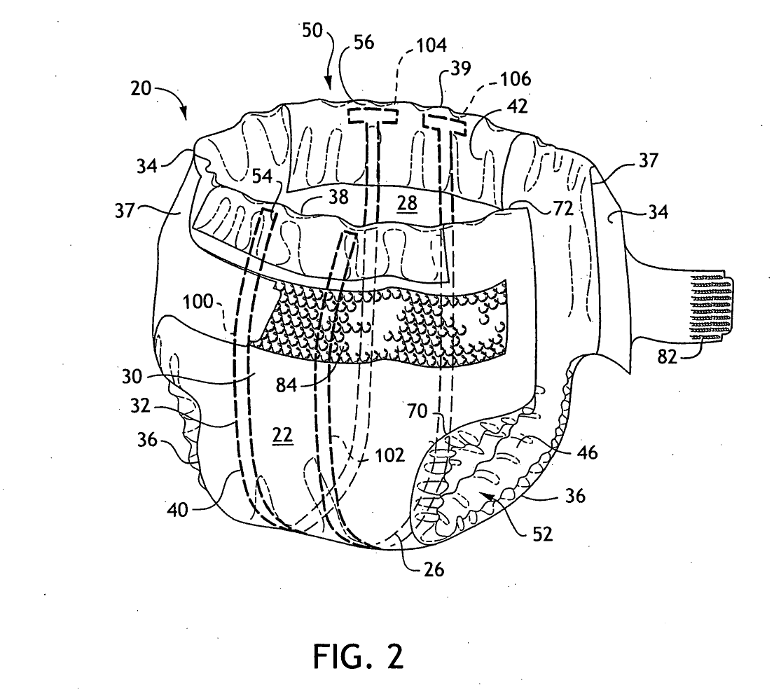

Referring to FIGS. 1 and 2, for exemplary purposes, a wetness sensing absorbent article 20 is shown. The wetness sensing absorbent article 20 may or may not be disposable. It is understood that the present invention is suitable for use with various other wetness sensing absorbent articles intended for personal wear, including but not limited to diapers, training pants, swim pants, feminine hygiene products, incontinence products, medical garments, surgical pads and bandages, other personal care or health care garments, and the like without departing from the scope of the present invention.

By way of illustration only, various materials and methods for constructing wetness sensing absorbent articles such as the wetness sensing absorbent article 20 of the various aspects of the present invention are disclosed in U.S. Pat. No. 4,798,603 issued Jan. 17, 1989, to Meyer et al.; U.S. Pat. No. 5,176,672 issued Jan. 5, 1993, to Bruemmer et al., U.S. Pat. No. 5,509,915 issued Apr. 23, 1996 to Hanson et al., U.S. Pat. No. 5,993,433 issued Nov. 30, 1999 to St. Louis et al., and U.S. Pat. No. 6,248,097 issued Jun. 19, 2001 to Beitz et al., PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al., and U.S. Pat. No. 6,645,190 issued Nov. 11, 2003 to Olson et al. which are incorporated herein by reference to the extent they are consistent (i.e., not in conflict) herewith.

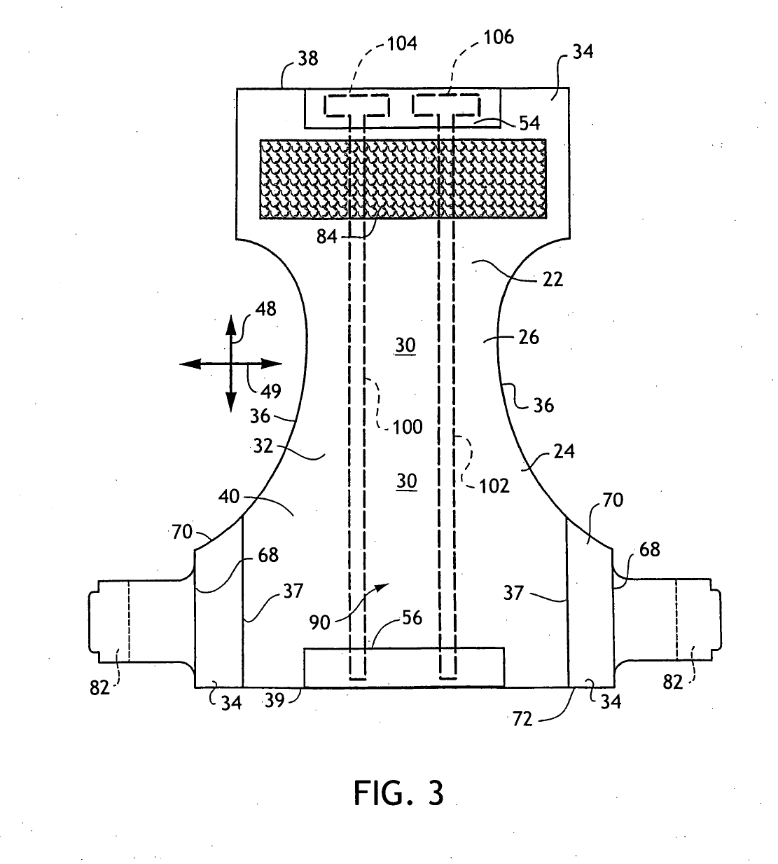

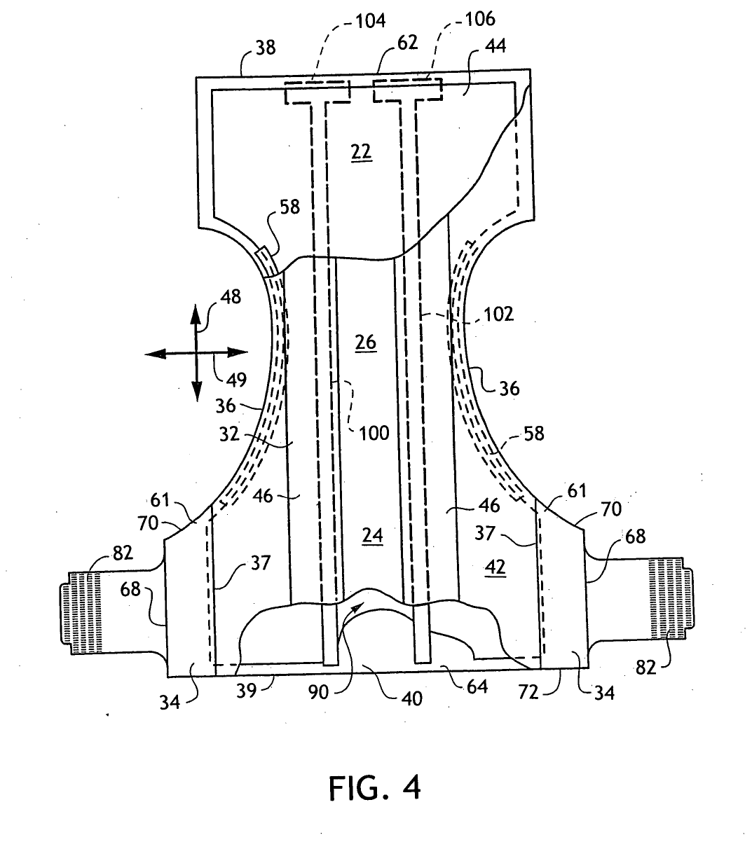

A wetness sensing absorbent article 20 is representatively illustrated in FIG. 1 in a partially fastened condition. The wetness sensing absorbent article 20 shown in FIGS. 1 and 2 is also represented in FIGS. 3 and 4 in an opened and unfolded state. Specifically, FIG. 3 is a plan view illustrating the exterior side of the wetness sensing absorbent article 20, while FIG. 4 illustrates the interior side of the wetness sensing absorbent article 20. As shown in FIGS. 3 and 4, the wetness sensing absorbent article 20 defines a longitudinal direction 48 that extends from the front of the article when worn to the back of the article. Opposite to the longitudinal direction 48 is a lateral direction 49.

The wetness sensing absorbent article 20 defines a pair of longitudinal end regions, otherwise referred to herein as a front region 22 and a back region 24, and a center region, otherwise referred to herein as a crotch region 26, extending longitudinally between and interconnecting the front and back regions 22, 24. The wetness sensing absorbent article 20 also defines an inner surface 28 adapted in use (e.g., positioned relative to the other components of the article 20) to be disposed toward the wearer, and an outer surface 30 opposite the inner surface. The front and back regions 22, 24 are those portions of the wetness sensing absorbent article 20, which when worn, wholly or partially cover or encircle the waist or mid-lower torso of the wearer. The crotch region 26 generally is that portion of the wetness sensing absorbent article 20 which, when worn, is positioned between the legs of the wearer and covers the lower torso and crotch of the wearer. The wetness sensing absorbent article 20 has a pair of laterally opposite side edges 36 and a pair of longitudinally opposite waist edges, respectively designated front waist edge 38 and back waist edge 39.

The illustrated wetness sensing absorbent article 20 includes a chassis 32 that, in this embodiment, encompasses the front region 22, the back region 24, and the crotch region 26. Referring to FIGS. 1-4, the chassis 32 includes an outer cover 40 and a bodyside liner 42 (FIGS. 1 and 4) that may be joined to the outer cover 40 in a superimposed relation therewith by adhesives, ultrasonic bonds, thermal bonds or other conventional techniques. Referring to FIG. 4, the liner 42 may suitably be joined to the outer cover 40 along the perimeter of the chassis 32 to form a front waist seam 62 and a back waist seam 64. As shown in FIG. 4, the liner 42 may suitably be joined to the outer cover 40 to form a pair of side seams 61 in the front region 22 and the back region 24. The liner 42 can be generally adapted, i.e., positioned relative to the other components of the article 20, to be disposed toward the wearer's skin during wear of the wetness sensing absorbent article. The chassis 32 may further include an absorbent structure 44 particularly shown in FIG. 4 disposed between the outer cover 40 and the bodyside liner 42 for absorbing liquid body exudates exuded by the wearer, and may further include a pair of containment flaps 46 secured to the bodyside liner 42 for inhibiting the lateral flow of body exudates.

The elasticized containment flaps 46 as shown in FIG. 4 define a partially unattached edge which assumes an upright configuration in at least the crotch region 26 of the wetness sensing absorbent article 20 to form a seal against the wearer's body. The containment flaps 46 can extend longitudinally along the entire length of the chassis 32 or may extend only partially along the length of the chassis. Suitable constructions and arrangements for the containment flaps 46 are generally well known to those skilled in the art and are described in U.S. Pat. No. 4,704,116 issued Nov. 3, 1987 to Enloe, which is incorporated herein by reference.

To further enhance containment and/or absorption of body exudates, the wetness sensing absorbent article 20 may also suitably include leg elastic members 58 (FIG. 4), as are known to those skilled in the art. The leg elastic members 58 can be operatively joined to the outer cover 40 and/or the bodyside liner 42 and positioned in the crotch region 26 of the wetness sensing absorbent article 20.

The leg elastic members 58 can be formed of any suitable elastic material. As is well known to those skilled in the art, suitable elastic materials include sheets, strands or ribbons of natural rubber, synthetic rubber, or thermoplastic elastomeric polymers. The elastic materials can be stretched and adhered to a substrate, adhered to a gathered substrate, or adhered to a substrate and then elasticized or shrunk, for example with the application of heat, such that elastic retractive forces are imparted to the substrate. In one particular aspect, for example, the leg elastic members 58 may include a plurality of dry-spun coalesced multifilament spandex elastomeric threads sold under the trade name LYCRA and available from Invista, Wilmington, Del., U.S.A.

In some embodiments, the wetness sensing absorbent article 20 may further include a surge management layer (not shown) which may be optionally located adjacent the absorbent structure 44 and attached to various components in the article 20 such as the absorbent structure 44 or the bodyside liner 42 by methods known in the art, such as by using an adhesive. A surge management layer helps to decelerate and diffuse surges or gushes of liquid that may be rapidly introduced into the absorbent structure of the article. Desirably, the surge management layer can rapidly accept and temporarily hold the liquid prior to releasing the liquid into the storage or retention portions of the absorbent structure. Examples of suitable surge management layers are described in U.S. Pat. No. 5,486,166; and U.S. Pat. No. 5,490,846. Other suitable surge management materials are described in U.S. Pat. No. 5,820,973. The entire disclosures of these patents are hereby incorporated by reference herein to the extent they are consistent (i.e., not in conflict) herewith.

As shown in FIGS. 1-4, the wetness sensing absorbent article 20 further includes a pair of opposing elastic side panels 34 that are attached to the back region of the chassis 32. As shown particularly in FIGS. 1 and 2, the side panels 34 may be stretched around the waist and/or hips of a wearer in order to secure the garment in place. As shown in FIGS. 3 and 4, the elastic side panels are attached to the chassis along a pair of opposing longitudinal edges 37. The side panels 34 may be attached or bonded to the chassis 32 using any suitable bonding technique. For instance, the side panels 34 may be joined to the chassis by adhesives, ultrasonic bonds, thermal bonds, or other conventional techniques.

In an alternative embodiment, the elastic side panels may also be integrally formed with the chassis 32. For instance, the side panels 34 may comprise an extension of the bodyside liner 42, of the outer cover 40, or of both the bodyside liner 42 and the outer cover 40.

In the embodiments shown in the figures, the side panels 34 are connected to the back region of the wetness sensing absorbent article 20 and extend over the front region of the article when securing the article in place on a user. It should be understood, however, that the side panels 34 may alternatively be connected to the front region of the article 20 and extend over the back region when the article is donned.

With the wetness sensing absorbent article 20 in the fastened position as partially illustrated in FIGS. 1 and 2, the elastic side panels 34 may be connected by a fastening system 80 to define a 3-dimensional wetness sensing absorbent article configuration having a waist opening 50 and a pair of leg openings 52. The waist opening 50 of the article 20 is defined by the waist edges 38 and 39 which encircle the waist of the wearer.

In the embodiments shown in the figures, the side panels are releasably attachable to the front region 22 of the article 20 by the fastening system. It should be understood, however, that in other embodiments the side panels may be permanently joined to the chassis 32 at each end. The side panels may be permanently bonded together, for instance, when forming a training pant or absorbent swimwear.

The elastic side panels 34 each have a longitudinal outer edge 68, a leg end edge 70 disposed toward the longitudinal center of the wetness sensing absorbent article 20, and waist end edges 72 disposed toward a longitudinal end of the wetness sensing absorbent article. The leg end edges 70 of the wetness sensing absorbent article 20 may be suitably curved and/or angled relative to the lateral direction 49 to provide a better fit around the wearer's legs. However, it is understood that only one of the leg end edges 70 may be curved or angled, such as the leg end edge of the back region 24, or alternatively, neither of the leg end edges may be curved or angled, without departing from the scope of the present invention. As shown in FIG. 4, the outer edges 68 are generally parallel to the longitudinal direction 48 while the waist end edges 72 are generally parallel to the transverse axis 49. It should be understood, however, that in other embodiments the outer edges 68 and/or the waist edges 72 may be slanted or curved as desired. Ultimately, the side panels 34 are generally aligned with a waist region 90 of the chassis.

The fastening system 80 may include laterally opposite first fastening components 82 adapted for refastenable engagement to corresponding second fastening components 84. In the embodiment shown in the figures, the first fastening component 82 is located on the elastic side panels 34, while the second fastening component 84 is located on the front region 22 of the chassis 32. In one aspect, a front or outer surface of each of the fastening components 82, 84 includes a plurality of engaging elements. The engaging elements of the first fastening components 82 are adapted to repeatedly engage and disengage corresponding engaging elements of the second fastening components 84 to releasably secure the article 20 in its three-dimensional configuration.

The fastening components 82, 84 may be any refastenable fasteners suitable for wetness sensing absorbent articles, such as adhesive fasteners, cohesive fasteners, mechanical fasteners, or the like. In particular aspects the fastening components include mechanical fastening elements for improved performance. Suitable mechanical fastening elements can be provided by interlocking geometric shaped materials, such as hooks, loops, bulbs, mushrooms, arrowheads, balls on stems, male and female mating components, buckles, snaps, or the like.

In the illustrated aspect, the first fastening components 82 include hook fasteners and the second fastening components 84 include complementary loop fasteners. Alternatively, the first fastening components 82 may include loop fasteners and the second fastening components 84 may be complementary hook fasteners. In another aspect, the fastening components 82, 84 can be interlocking similar surface fasteners, or adhesive and cohesive fastening elements such as an adhesive fastener and an adhesive-receptive landing zone or material; or the like. One skilled in the art will recognize that the shape, density and polymer composition of the hooks and loops may be selected to obtain the desired level of engagement between the fastening components 82, 84. Suitable fastening systems are also disclosed in the previously incorporated PCT Patent Application WO 00/37009 published Jun. 29, 2000 by A. Fletcher et al. and the previously incorporated U.S. Pat. No. 6,645,190 issued Nov. 11, 2003 to Olson et al.

In the embodiment shown in the figures, the fastening components 82 are attached to the side panels 34 along the edges 68. In this embodiment, the fastening components 82 are not elastic or extendable. In other embodiments, however, the fastening components may be integral with the side panels 34. For example, the fastening components may be directly attached to the side panels 34 on a surface thereof.

In addition to possibly having elastic side panels, the wetness sensing absorbent article 20 may include various waist elastic members for providing elasticity around the waist opening. For example, as shown in the figures, the wetness sensing absorbent article 20 can include a front waist elastic member 54 and/or a back waist elastic member 56.

As described above, the present disclosure is particularly directed to incorporating a body fluid indicating system. One such system is described below. Other systems include a wetness liner such as that described in U.S. Pat. No. 6,658,432 to Underhill et al., a temperature system, a system in which graphics fade or appear, and any other suitable body fluid indicating system.

One such body fluid indicating system is the wetness indicating system described herein. In this regard, as shown in FIGS. 1-4, the wetness sensing absorbent article 20 includes a first conductive element 100 spaced from a second conductive element 102. In this embodiment, the conductive elements extend from the front region 22 of the wetness sensing absorbent article to the back region 24 without intersecting. The conductive elements 100 and 102 can comprise any suitable conductive material, such as a conductive thread or a conductive foil for example include 112-S silver metallic conductive paste (ink) from Electroscience Laboratories, Inc. and conductive foil described in U.S. Pat. No. 6,417,455 issued Jul. 9, 2002 to Zein et. Al. The first conductive element 100 may not intersect the second conductive element 102 in order to form an open circuit that may be closed, for instance, when a conductive fluid is positioned in between the conductive elements. In other embodiments, however, the first conductive element 100 and the second conductive element 102 may be connected to a sensor within the chassis. The sensor may be used to sense changes in temperature or may be used to sense the presence of a particular substance, such as a metabolite.

In the embodiment shown in FIG. 1, the conductive elements 100 and 102 extend the entire length of the wetness sensing absorbent article 20. It should be understood, however, that in other embodiments the conductive elements may extend only to the crotch region 26 or may extend to any particular place in the wetness sensing absorbent article where a body fluid is intended to be sensed.

The conductive elements 100 and 102 may be incorporated into the chassis 32 at any suitable location as long as the conductive elements are positioned so as to contact a body fluid that is absorbed by the wetness sensing absorbent article 20. In this regard, the conductive elements 100 and 102 generally lie inside the outer cover 40. In fact, in one embodiment, the conductive elements 100 and 102 may be attached or laminated to the inside surface of the outer cover 40 that faces the absorbent structure 44. Alternatively, however, the conductive elements 100 and 102 may be positioned on the absorbent structure 44 or positioned on the liner 42.

The conductive element 100 and 102 may be connected directly to a signaling device, either through direct or indirect contact. The first conductive element 100 may be attached to a first conductive pad member 104, while the second conductive element 102 may be connected to a second conductive pad member 106. The pad members 104 and 106 may be provided for making a reliable connection between the open circuit formed by the conductive elements to a signaling device that is intended to be installed on the chassis by the consumer or manufacturer. The pad members 104 and 106 may create a target zone for attaching the signaling device and the conductive leads or elements.

The conductive pad members 104 and 106 may have a relatively large surface area in relation to the conductive elements 100 and 102. For example, the conductive pad members 104 and 106 may have a surface area of at least 1 cm², at least 2 cm², and, in one embodiment, at least 3 cm². For instance, in one embodiment, the surface area of each pad member may be from about 2 cm² to about 4 cm².

The position of the conductive pad members 104 and 106 on the wetness sensing absorbent article 20 can vary depending upon where it is desired to mount the signaling device. For instance, in FIGS. 1, 3 and 4, the conductive pad members 104 and 106 are positioned in the front region 22 along the waist opening of the article. In FIG. 2, on the other hand, the conductive pad members 104 and 106 are positioned in the back region 24 along the waist opening of the article. It should be appreciated, however, that in other embodiments, the wetness sensing absorbent article 20 may include conductive pad members being positioned at each end of each conductive element 100 and 102. In still other embodiments, it should be understood that the pad members may be located along the side of the article or towards the crotch region of the article.

The position of the conductive pad members 104 and 106 within the multiple layers of the chassis 32 may also vary depending upon where it is desired to connect the signaling device and the type of attachment mechanism used to make a connection with the signaling device. As described above, the pad members 104 and 106 are electrically connected to the conductive elements 100 and 102. Thus, in one embodiment, the pad members 104 and 106 are positioned below (toward the body side) at least one layer of the outer cover 40. Positioning the pad members 104 and 106 below at least one layer of material may provide various advantages in some embodiments. For instance, locating the pad members 104 and 106 below at least one layer of material within the chassis 32 protects the pad members during shipping and storage and from forming a short circuit during use especially if the pad members are located adjacent one another. Another benefit to placing the pad members under at least one layer of material is the ability to easily manufacture the wetness sensing absorbent article 20 at high machine speeds.

It should be understood, however, that in other embodiments the conductive pad members 104 and 106 may be positioned at an exterior surface of the chassis 32. For instance, the pad members 104 and 106 may be positioned on the outside surface or on the inside surface as desired.

The remaining materials used to form the wetness sensing absorbent article 20 may vary depending upon the particular application and the particular product being produced.

The outer cover 40, for instance, may be breathable and/or may be liquid impermeable. The outer cover 40 may be constructed of a single layer, multiple layers, laminates, spunbond fabrics, films, meltblown fabrics, elastic netting, microporous webs, bonded card webs or foams provided by elastomeric or polymeric materials. The outer cover 40, for instance, can be a single layer of a liquid impermeable material, or alternatively can be a multi-layered laminate structure in which at least one of the layers is liquid impermeable. In other embodiments, however, it should be understood that the outer cover may be liquid permeable. In this embodiment, for instance, the wetness sensing absorbent article may contain an interior liquid barrier layer.

For instance, the outer cover 40 can include a liquid permeable outer layer and a liquid impermeable inner layer that are suitably joined together by a laminate adhesive, ultrasonic bonds, thermal bonds, or the like. Suitable laminate adhesives, which can be applied continuously or intermittently as beads, a spray, parallel swirls, or the like, can be obtained from Bostik, Inc., of Wauwatosa, Wis., U.S.A., or from National Starch and Chemical Company, Bridgewater, N.J., U.S.A. The liquid permeable outer layer can be any suitable material and is desirably one that provides a generally cloth-like texture. One example of such a material is a 20 gsm (grams per square meter) spunbond polypropylene nonwoven web. The outer layer may also be made of those materials of which the liquid permeable bodyside liner 42 is made.

The inner layer of the outer cover 40 can be both liquid and vapor impermeable, or it may be liquid impermeable and vapor permeable. The inner layer can be manufactured from a thin plastic film, although other flexible liquid impermeable materials may also be used. The inner layer, or the liquid impermeable outer cover 40 when a single layer, prevents waste material from wetting articles, such as bed sheets and clothing, as well as the wearer and caregiver. A suitable liquid impermeable film for use as a liquid impermeable inner layer, or a single layer liquid impermeable outer cover 40, is a 0.02 millimeter polyethylene film commercially available from Pliant Corporation of Schaumburg, Ill., U.S.A.

The bodyside liner 42 is suitably compliant, soft-feeling, and non-irritating to the wearer's skin. The bodyside liner 42 is also sufficiently liquid permeable to permit liquid body exudates to readily penetrate through its thickness to the absorbent structure 44. A suitable bodyside liner 42 may be manufactured from a wide selection of web materials, such as porous foams, reticulated foams, apertured plastic films, woven and non-woven webs, or a combination of any such materials. For example, the bodyside liner 42 may include a meltblown web, a spunbonded web, or a bonded-carded-web composed of natural fibers, synthetic fibers or combinations thereof. The bodyside liner 42 may be composed of a substantially hydrophobic material, and the hydrophobic material may optionally be treated with a surfactant or otherwise processed to impart a desired level of wettability and hydrophilicity.

The absorbent structure 44 may be disposed between the outer cover 40 and the bodyside liner 42. The absorbent structure 44 can be any structure or combination of components which are generally compressible, conformable, non-irritating to a wearer's skin, and capable of absorbing and retaining liquids and certain body wastes. For example, the absorbent structure 44 may include an absorbent web material of cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In a particular aspect, the absorbent web material is a matrix of cellulosic fluff and superabsorbent hydrogel-forming particles. The cellulosic fluff may include a blend of wood pulp fluff. One preferred type of fluff is identified with the trade designation CR 1654, available from Bowater of Greenville, S.C., USA, and is a bleached, highly absorbent sulfate wood pulp containing primarily southern soft wood fibers. The absorbent materials may be formed into a web structure by employing various conventional methods and techniques. For example, the absorbent web may be formed with a dry-forming technique, an air forming technique, a wet-forming technique, a foam-forming technique, or the like, as well as combinations thereof. Methods and apparatus for carrying out such techniques are well known in the art. Furthermore, the absorbent structure may itself encompass multiple layers in the Z direction. Such multiple layers may take advantage of differences in absorbency capacity, such as by placing a lower capacity absorbent material layer closer to the liner 42 and a higher capacity absorbent material closer to the outer cover layer 40. Likewise, discrete portions of an absorbent single-layered structure may encompass higher capacity absorbents, and other discrete portions of the structure may encompass lower capacity absorbents.

As a general rule, the superabsorbent material is present in the absorbent web in an amount of from about 0 to about 90 weight percent based on total weight of the web. The web may have a density within the range of about 0.10 to about 0.60 grams per cubic centimeter.

Superabsorbent materials are well known in the art and can be selected from natural, synthetic, and modified natural polymers and materials. The superabsorbent materials can be inorganic materials, such as silica gels, or organic compounds, such as crosslinked polymers. Typically, a superabsorbent material is capable of absorbing at least about 10 times its weight in liquid, and desirably is capable of absorbing more than about 25 times its weight in liquid. Suitable superabsorbent materials are readily available from various suppliers. For example, SXM 9394, and Favor 9543 superabsorbents are available from DeGussa Superabsorbers.

After being formed or cut into a desired shape, the absorbent web material may be wrapped or encompassed by a suitable tissue or meltblown web or the like wrap sheet that aids in maintaining the integrity and shape of the absorbent structure 44.

The absorbent web material may also be a coform material. The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic absorbent materials, treated polymeric staple fibers and the like. Any of a variety of synthetic polymers may be utilized as the melt-spun component of the coform material. For instance, in certain aspects, thermoplastic polymers can be utilized. Some examples of suitable thermoplastics that can be utilized include polyolefins, such as polyethylene, polypropylene, polybutylene and the like; polyamides; and polyesters. In one aspect, the thermoplastic polymer is polypropylene. Some examples of such coform materials are disclosed in U.S. Pat. No. 4,100,324 to Anderson, et al.; U.S. Pat. No. 5,284,703 to Everhart, et al.; and U.S. Pat. No. 5,350,624 to Georger, et al.; which are incorporated herein by reference to the extent they are consistent (i.e., not in conflict) herewith.

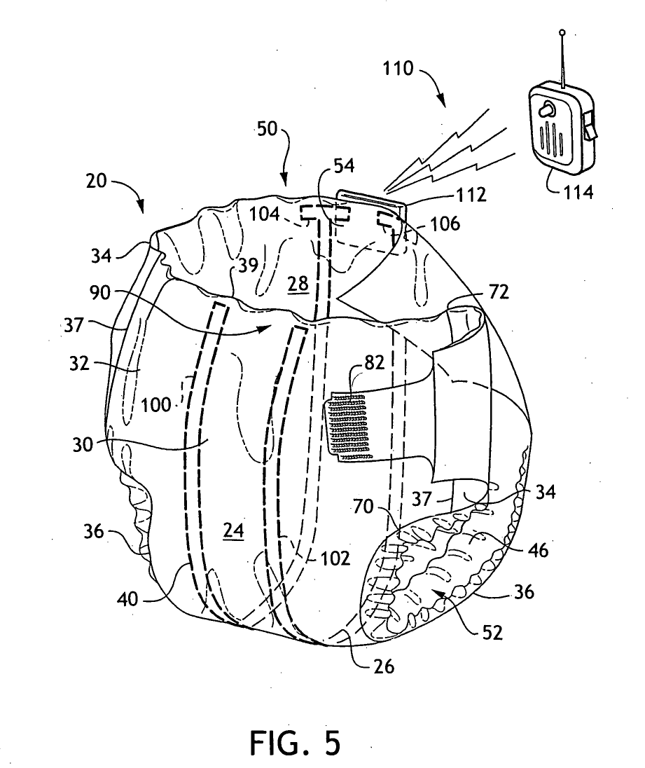

Referring to FIG. 5, for exemplary purposes, a signaling device 110 (as depicted by ref. numerals 112 and 114) is shown attached to the conductive pad members 104 and 106. As shown, in this embodiment, the signaling device generally 110 includes a transmitter 112 and a receiver 114. The transmitter 112 includes a pair of opposing terminals that are electrically connected to the corresponding conductive elements. When a body fluid is present in the wetness sensing absorbent article 20, the open circuit formed by the conductive elements 100 and 102 is closed which, in turn, activates the signaling device 110. In particular, in this embodiment, the transmitter 112 sends a wireless signal to the receiver 114 which then indicates to a user that a body fluid is present in the wetness sensing absorbent article 20.

The signaling device 110 can emit an audible signal or a visual signal in order to indicate to the user that the circuit has been closed. The audible signal, for instance, may be as simple as one or more beeps to perhaps emitting a musical tune. Similarly, if the signaling device 110 issues a visible signal, the visible signal may comprise a few lights or an interactive display. In still another embodiment, the receiver 114 of the signaling device 110 may be configured to vibrate when the circuit within the wetness sensing absorbent article is closed.

In the embodiment shown in FIG. 5, the signaling device 110 includes a transmitter 112 in combination with a receiver 114. It should also be understood, however, that the signaling device may comprise a single unit that remains attached to the wetness sensing absorbent article 20. For example, the signaling device may be mounted on the wetness sensing absorbent article and issue a visible signal and/or an audible signal from the article itself.

In various aspects of the present invention, the absorbent article 20 may include additional features such as those disclosed in co-pending and co-assigned U.S. patent application Ser. No. 11/303,283 to Long, et al. and entitled “Garments With Easy-To-Use Signaling Device”; and U.S. patent application Ser. No. 11/215,937 to Ales, et al. and entitled “Method of Detecting the Presence of an Insult in an Absorbent Article and Device for Detecting the Same”; which are incorporated herein by reference to the extent they are consistent (i.e., not in conflict) herewith. For example, the wetness sensing absorbent article may also include other wetness sensing features such as fading ink, appearing ink, a wetness liner, or a cooling component.

The wetness sensing absorbent article 20 may be a part of a wetness sensing system 120. In one aspect of the present invention, the wetness sensing system 120 includes as wetness sensing components a wetness sensing absorbent article 20 and a signaling device 110 that is compatible with the wetness sensing absorbent article 20. The wetness sensing system 120 may also include as a wetness sensing component a receiver 114. The wetness sensing system 120 may also include as wetness sensing components a second signaling device or other signaling devices and/or wetness sensing absorbent articles that are also compatible with the wetness sensing absorbent article 20. In various aspects of the present invention, the wetness sensing system 120 may be configured to be used in toilet training a child, in addressing enuresis in a subject, or in monitoring incontinence in a subject, particularly an adult. In one aspect of the present invention, each signaling device manufactured and sold will be compatible with every wetness sensing absorbent article manufactured and sold for any application. For example, a wetness sensing absorbent article 20 may be manufactured and sold for toilet training purposes, and another wetness sensing absorbent article 20 may be manufactured and sold to address enuresis concerns. Likewise, the signaling device 110 and other signaling devices may be manufactured and sold that will both be compatible with both the toilet training and enuresis wetness sensing absorbent articles.

In one aspect of the present invention, the wetness sensing system may be enhanced by producing different types of signaling devices beyond the signaling device 110, or by producing wetness sensing absorbent articles that are also compatible with the signaling devices.

In various aspects, the wetness sensing system 120 may include as wetness sensing components wetness sensing informational items such as instructions in the use of the product and tips for toilet training, enuresis control, or incontinence control. As used herein, the term “wetness sensing informational item” refers to objects that are provided in addition to wetness sensing absorbent articles, are adapted to communicate information to the user and/or consumer of the wetness sensing absorbent article, and are associated with individual components of the wetness sensing system 120. Examples of wetness sensing informational items include cards, paper, electronic media, printing on the packaging, or other suitable media capable of storing and conveying information.

In various aspects, the wetness sensing informational items associated with the wetness sensing system components may be adapted to appeal to the specific category of user and/or purchaser to which the wetness sensing absorbent article is adapted. The wetness sensing informational items may be adapted, for example, by providing information likely to be of interest to a given category of user and/or purchaser.

For example, a wetness sensing absorbent article may be adapted for use by a caregiver for toilet training purposes. A wetness sensing informational item may be associated with the wetness sensing absorbent article that is adapted to interest caregivers. For example, the wetness sensing informational item may be a card containing information or instructions about children's health and hygiene, such as sleep habits, thumb sucking, teething, skin health, toilet training; questions to ask a child; jokes; and the like, and combinations thereof. The wetness sensing informational item may additionally or alternatively include addresses for web sites available on the Internet. The web sites may contain information related to issues of interest for caregivers and users of wetness sensing absorbent articles.

The wetness sensing informational item may additionally or alternatively include information describing activities that are suitable for caregivers and users of wetness sensing absorbent articles. The activities may be adapted for a child at a specific age, size and/or stage of development. For example, the activities may be adapted to promote interaction between the child and the caregiver.

The wetness sensing informational item may additionally or alternatively include information describing the benefits to be derived from using the wetness sensing system 120. This wetness sensing informational item would be part of a promotional plan emphasizing the customizability of the wetness sensing system 120 for the benefit of the consumer, caregiver, and/or user. This wetness sensing informational item would both explain the use of the various components of the wetness sensing system 120 as well as presenting the additional components that may be available and the various combinations that are possible to achieve different goals.

Keys to establishing a training or notification program to address a lack of urination control in a subject include consistency and confidence. Consistency is demonstrated by continuing the program without disruption once it is started. That consistency is enhanced by choosing products that are compatible and that are widely, consistently, and immediately available. Likewise, it is desirable to enhance confidence because confidence shown by a caregiver will transfer to a subject. Caregiver confidence can be enhanced by being aware of the options available, and by choosing products that are effective, reputable, supported, reasonably priced, and readily available. These concepts are each discussed in more detail below.

The consistency of a training or notification program is enhanced by providing compatible products. The caregiver may have one wetness sensing component but may have exhausted the supply of another wetness sensing component. The caregiver can be assured that, with compatible wetness sensing components, further wetness sensing components obtained will work with those already in the possession of the caregiver. Various wetness sensing components of the wetness sensing system 120 may be compatible with each other as described in co-assigned and co-pending U.S. patent application Ser. No. ______ and entitled, “An Array Of Wetness-Sensing Articles,” the entire disclosure of which is hereby incorporated by reference herein to the extent it is consistent (i.e., not in conflict) herewith.

Similarly, the consistency of a program is enhanced by providing wetness sensing components that are widely, consistently, and immediately available. The caregiver may desire to initiate a program and need a full range of wetness sensing components, or the caregiver may have one wetness sensing component but may have exhausted the supply of another wetness sensing component. The caregiver can be assured that, with widely, consistently, and immediately available wetness sensing components, further wetness sensing components can be obtained.

Various wetness sensing components may currently be available via the Internet or by telephone or mail order, but these products can be costly to obtain, slow to arrive, or both. Days may pass between a caregiver recognizing a need for wetness sensing components and having those wetness sensing components in hand. A training or notification program can easily be disrupted and any progress lost during that time. It is therefore in the nature of the wetness sensing components supporting such a training or notification program that they be immediately available to a caregiver to optimize the progress of such a program.

In addition, a wetness sensing system is unique in the respect that a caregiver will not typically have access to materials that may used as a substitute that will fill a gap in the supply of a wetness sensing component. A caregiver might typically have access to materials or actions that may substitute, however temporarily, for standard absorbent articles, but wetness sensing components must be replenished promptly to maintain progress in a training or notification program.

Further, component compatibility is not an issue for standard absorbent articles. One need only find an absorbent article that will generally fit. In an emergency situation, most any size can be made to fit. In the case of a wetness sensing system, one must find components that will work together, or a component that will work with other components one already has.

A wetness sensing system manufacturer with manufacturing facilities and a distribution network supplying a number of retail outlets will be able to make wetness sensing components easily and immediately available to the caregiver. Wetness sensing components may be made available at a network of retail outlets including department stores, big-box retailers, supermarkets, pharmacies, and/or convenience stores that the caregiver already visits on a regular basis. The caregiver knows wetness sensing components are immediately available close to home.

To effectively enhance the wide, consistent, and immediate availability of wetness sensing components, such wetness sensing components may be made available in at least 5 retail outlets. Consistent availability increases with the number of retail outlets carrying such wetness sensing components and ideally includes availability at 50, 200, 500, 1000, 1500, 2000, or even 2500 or more retail outlets.

For example, if wetness sensing components are made available in Wal-Mart stores, the wetness sensing components will be readily available to a large proportion of the United States. In his book The Wal-Mart Effect: How the World's Most Powerful Company Really Works—and How It's Transforming the American Economy, p. 5, (The Penguin Press, 2006), author Charles Fishman states that “[m]ore than half of all Americans live within five miles of a Wal-Mart store, less than a ten-minute drive away. Ninety percent of Americans live within fifteen miles of a Wal-Mart.” With wetness sensing components supplied to other retail outlets as well, almost every American would be within minutes of obtaining needed wetness sensing components.

The wetness sensing system manufacturer may enhance consistency further by supplying a geographically-distributed network of retail outlets. Wetness sensing components are made available in retail outlets throughout one or more cities, counties, states, countries, and or continents. The caregiver then knows wetness sensing components are immediately available close to home and virtually anywhere the caregiver may travel.

To effectively enhance the wide, consistent, and immediate availability of wetness sensing components, such wetness sensing components may be made available in at least 2 states of the United States. Consistent availability increases with the number of geographic locations carrying such wetness sensing components and ideally includes availability in 5, 10, 25, 30, 35, or even 40 or more states of the United States.

To further effectively enhance the wide, consistent, and immediate availability of wetness sensing components, such wetness sensing components may be made available in at least 2 countries. Consistent availability increases with the number of geographic locations carrying such wetness sensing components and ideally includes availability in 5, 10, 15, or even 20 or more countries.

To still further effectively enhance the wide, consistent, and immediate availability of wetness sensing components, such wetness sensing components may be made available on at least 2 continents. Consistent availability increases with the number of geographic locations carrying such wetness sensing components and ideally includes availability on 3, 4, 5, or even 6 or more continents.

In one aspect of the present invention, the wetness sensing system manufacturer may advantageously maintain a network of two or more distribution centers that may directly or indirectly receive wetness sensing components from a manufacturer for direct or indirect distribution to the retail outlets. In this manner, wetness sensing components should always be plentifully and promptly supplied to the retail outlets.

In addition to consistency, the potential success of a training or notification program is improved by enhancing the confidence of a caregiver. As described above, caregiver confidence can be enhanced by being aware of the options available, and by choosing products that are effective, reputable, supported, reasonably priced, and readily available.

In one aspect of the present invention, confidence is enhanced by making the caregiver aware of the options available including various to wetness sensing components, wetness sensing informational items, and program types. In one aspect of the present invention, caregiver awareness is increased by promoting the wetness sensing system in a variety of channels. In this use, the term channels refers to methods of advertising and information distribution. For example, the advertising and/or information on the wetness sensing system may be distributed on television, in print media, through direct mail pieces, on the Internet, or by demonstrations in retail outlets and other venues. Advertising and/or information may also be distributed in physician offices and hospitals, in health fairs or presentations, in schools, in child care facilities, or by any other suitable distribution channel. Caregiver awareness is maximized when such advertising and/or information distribution is advantageously provided by a wetness sensing system manufacturer or other entity with the experience and resources to make such advertising and/or information effective and widespread.

In addition, caregiver awareness is further maximized where the distribution of such advertising and/or information is advantageously geographically-distributed in much the same way, and with the same ideal distribution, as the retail outlets described above.

Caregiver confidence can also be enhanced by providing an effective and targeted wetness sensing system 120. The wetness sensing system 120 is advantageously provided by a wetness sensing system manufacturer with sufficient experience and resources to conduct or obtain detailed and extensive consumer use tests and clinicals to enable that manufacturer to design and manufacture a wetness sensing system of optimal effectiveness and includes features desired by caregivers. Additional input that will increase caregiver confidence in the wetness sensing system design and manufacture may be obtained through marketing and business analysis, through access to global trending and usage and efficacy data, and through the kinds of high product quality and safety standards available to an experienced manufacturer.

Caregiver confidence can also be enhanced by a making wetness sensing components available from a reputable manufacturer or by marketing the wetness sensing components under a reputable brand or trademark. For example, caregiver confidence is heightened when the wetness sensing components they are considering are manufactured by a wetness sensing system manufacturer with experience in manufacturing and selling absorbent articles. To effectively enhance caregiver confidence, wetness sensing components may be made available from a manufacturer with at least 5 years of experience in manufacturing and selling absorbent articles. Caregiver confidence increases with the absorbent article manufacturing experience of the wetness sensing component manufacturer and ideally includes experience of 10, 20, 30 or even 50 or more years.

In addition, such experience may be demonstrated to the caregiver by packaging a wetness sensing component in a package displaying a trademark of a company having experience in manufacturing and selling absorbent articles. To effectively enhance caregiver confidence, wetness sensing components may be packaged in a package displaying the trademark of a company with at least 5 years of experience in manufacturing and selling absorbent articles. Caregiver confidence increases with the absorbent article manufacturing experience of the wetness sensing component manufacturer and ideally includes experience of 10, 20, 30 or even 50 or more years.

Similarly, such experience may be demonstrated to the caregiver by selling a wetness sensing component under a brand having a significant market share of absorbent article sales in the United States. To effectively enhance caregiver confidence, wetness sensing components may be sold under a brand with a market share of at least 5 percent of the United States absorbent article market. Caregiver confidence increases with the brand's market share and ideally includes market shares of 10, 20, 30, 50 or even 70 percent.

Also similarly, such experience may be demonstrated to the caregiver by packaging a wetness sensing component in a package displaying a trademark or brand having a significant total brand awareness or brand recognition rate and a significant brand equity score with respect to providing absorbent articles. In this use, total brand awareness is the sum of aided and unaided brand awareness and is equivalent to brand recognition rate. To effectively enhance caregiver confidence, wetness sensing components may be packaged in a package displaying a trademark or brand having a brand recognition rate of at least 20 percent. Caregiver confidence increases with the brand recognition rate and ideally includes brand recognition rates of 30, 40, 50, 60, 70, 80, 90, or even 95 percent.

In addition, to effectively enhance caregiver confidence, wetness sensing components may be packaged in a package displaying a trademark or brand having a brand equity score equivalent to the brand equity score of the market share leader in the type of absorbent articles upon which the wetness sensing absorbent article is based. For example, if the wetness sensing absorbent article is based on a training pant, then the wetness sensing components are packaged in a package displaying a trademark or brand having a brand equity score equivalent to the brand equity score of the market share leader in training pants. Caregiver confidence increases with the brand equity score and includes brand equity scores at 5, 10, 20, 30, 50, 75, 90, 95, or even 100 percent of the brand equity score of the market share leader in the type of absorbent articles upon which the wetness sensing absorbent article is based.

Caregiver confidence can also be enhanced by a making wetness sensing components available from a manufacturer of sufficient experience and resources to support the wetness sensing components with in-house experts. Such experts may include experts in product design, manufacturing processes, marketing, clinical testing, use testing, human health, child development, psychology, nursing, and physician assistants as non-limiting examples.

Caregiver confidence can also be enhanced by a making wetness sensing components available at a reasonable price. A wetness sensing system manufacturer of sufficient experience and resources may advantageously provide reasonably-priced wetness sensing components by, for example, optimizing manufacturing processes and raw material sourcing, and using negotiating power in providing products to distributors and retailers. The wide scale manufacturing capabilities, high volume production, state-of-the-art processing techniques, low cost material and production, and other means available to a wetness sensing system manufacturer of sufficient size and resources allow that manufacturer to optimize the cost at which wetness sensing components can be provided. In addition, these means also allow the manufacturer to produce large numbers of consistent wetness sensing components and to make the wetness sensing components widely and affordably available.

A further benefit to consumers accrues from confidence, whether conscious or subconscious, gained by acquiring wetness sensing components from a well-known and reputably innovative company in that the consumer knows that if one product fails to have the effect of aiding the caregiver in a particular situation, the company will have other products or product improvements that will be effective in that situation. A well-known and reputably innovative company will not want to risk the reputation of its brands by producing an ineffective product or array of products. The caregiver will in essence have a safety net of support in seeking effective solutions. The caregiver can be supplied with increasingly sophisticated and targeted solutions.

Caregiver confidence can also be enhanced by a making wetness sensing components widely, consistently, and immediately available, as described herein. A caregiver will have confidence in a wetness sensing system if that caregiver knows that any component of that system will be almost always available, wherever and whenever the caregiver may seek them.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged either in whole or in part. Furthermore, those of ordinary skill in the art will appreciate that the foregoing description is by way of example only, and is not intended to limit the invention so further described in such appended claims.

What is claimed:

1. A method for assisting a caregiver who is addressing a lack of urination control in a subject, the method comprising:
manufacturing a plurality of wetness sensing systems, each including a plurality of wetness sensing components;
providing the wetness sensing systems to a plurality of geographically-distributed retail outlets; and
increasing consumer awareness of the wetness sensing systems by promoting the wetness sensing systems through a plurality of channels.

2. The method of claim 1, wherein the wetness sensing components include at least two of a wetness sensing absorbent article, a signaling device, wetness sensing instructions, and a receiver.

3. The method of claim 1, wherein providing the wetness sensing systems includes providing the wetness sensing systems to at least 25 retail outlets.

4. The method of claim 1, wherein providing the wetness sensing systems includes providing the wetness sensing systems to retail outlets in at least two states.

5. The method of claim 1, wherein providing the wetness sensing systems includes providing the wetness sensing systems to retail outlets in at least two countries.

6. The method of claim 1, wherein providing the wetness sensing systems includes providing the wetness sensing systems to retail outlets on at least two continents.

7. The method of claim 1, wherein manufacturing the wetness sensing systems includes manufacturing the wetness sensing systems in a quantity sufficient to effectively minimize the as-manufactured cost of the wetness sensing systems.

8. The method of claim 1, wherein manufacturing the wetness sensing systems includes manufacturing at least one wetness sensing component of the wetness sensing systems in a manufacturing facility controlled by a company having at least 5 years of experience with manufacturing and selling absorbent articles.

9. The method of claim 1, further comprising packaging a wetness sensing component in a package displaying a trademark having a brand recognition rate greater than 20 percent.

10. The method of claim 1, wherein the wetness sensing components include a wetness sensing absorbent article, and further comprising packaging a wetness sensing component in a package displaying a trademark having a brand equity score at least at 5 percent of that of the market share leader in the type of absorbent articles upon which the wetness sensing absorbent article is based.

11. The method of claim 1, further comprising packaging a wetness sensing component in a package displaying a brand, wherein the brand has a market share of at least 10 percent of the United States absorbent article market.

12. The method of claim 1, further comprising packaging a wetness sensing component in a package displaying a trademark of a company having at least 10 years of experience with manufacturing and selling absorbent articles.

13. The method of claim 1, further comprising transferring a plurality of wetness sensing systems to a plurality of geographically-distributed distribution centers.

14. A system for increasing consumer convenience in acquiring wetness sensing components, the system comprising:
a first manufacturing facility for producing wetness sensing absorbent articles;
a second manufacturing facility for producing signaling devices compatible with the wetness sensing absorbent articles; and
at least 25 retail outlets, each receiving at least one of the wetness sensing absorbent articles and at least one of the signaling devices, wherein a wetness sensing absorbent article purchased at one retail outlet is compatible with a signaling device sold at another retail outlet.

15. The system of claim 14, wherein each wetness sensing absorbent article includes a conductor and a means for connecting a signaling device to the wetness sensing absorbent article.

16. The system of claim 14, further comprising a plurality of distribution centers for receiving wetness sensing components from the first or second manufacturing facilities.

17. The system of claim 14, further comprising a marketing program for increasing consumer awareness of the wetness sensing components by promoting the wetness sensing components through a plurality of channels.

18. The system of claim 14, wherein the retail outlets are geographically-distributed.
